(19) United States
(12) Plant Patent Application Publication
(10) Pub. No.: US 2015/0289429 P1
(43) Pub. Date: Oct. 8, 2015

(54) Strawberry plant named 'NJ00-48-03'

(50) ;

(71) Applicant: Rutgers, The State University, New Brunswick, NJ (US)

(72) Inventors: Gojko J. Jelenkovic, Piscataway, NJ (US); Laurie P. Lutz, Rockville, MD (US); Peter J. Nitzsche, Long Valley, NJ (US); William T. Hlubik, Bordentown, NJ (US)

(73) Assignee: Rutgers, The State University, New Brunswick, NJ (US)

(21) Appl. No.: 13/999,928

(22) Filed: Apr. 4, 2014

(51) Int. Cl. A01H 5/00

(52) U.S. Cl. PLT/208

(57) ABSTRACT

A new and distinct cultivar of strawberry plant (Fragaria×annanassa) named ‘NJ00-48-3’. The new strawberry plant is distinguished by its ability to produce large, uniform, and firm fruit with excellent flavor. This vigorous plant has an upright form, is a short day cultivar distinguished by its elongated petioles, upright plant growth and fruits, with a uniform mild red color, shiny in appearance and conical in shape.

### CROSS REFERENCE TO RELATED APLICATIONS

NONE

### STATEMENT REGARDING FEDERALLY SPONSORED RESEARCH OR DEVELOPMENT

NONE

### LATIN NAME OF GENUS AND SPECIES OF THE PLANT CLAIMED

Fragaria annanassa

### VARIETY DENOMINATION

‘NJ00-48-03’

### BACKGROUND OF THE NEW PLANT

A new and distinct cultivar of strawberry named ‘NJ00-48-3’ is a short day cultivar distinguished by its elongated petioles, upright plant growth and large fruits, with a uniform red color, shiny in appearance and conical in shape with exceptional flavor. The cultivar is well adapted to high density plasti-culture growing systems and has been shown to perform well in the eastern United States. This new variety should be of commercial value, particularly for farmers with direct markets, due to its unique growth characteristics and excellent fruit production and flavor.

### BRIEF DESCRIPTION OF THE PHOTOGRAPHS

FIG. 1 illustrates the characteristics (shape, size and coloring) of typical ripened fruit from the ‘NJ00-48-3’ plant. The fruits are mostly conical in shape, the primary and secondary fruits are a bit larger than the tertiary fruits. The fruits are a mild to deep red color when fully ripe.

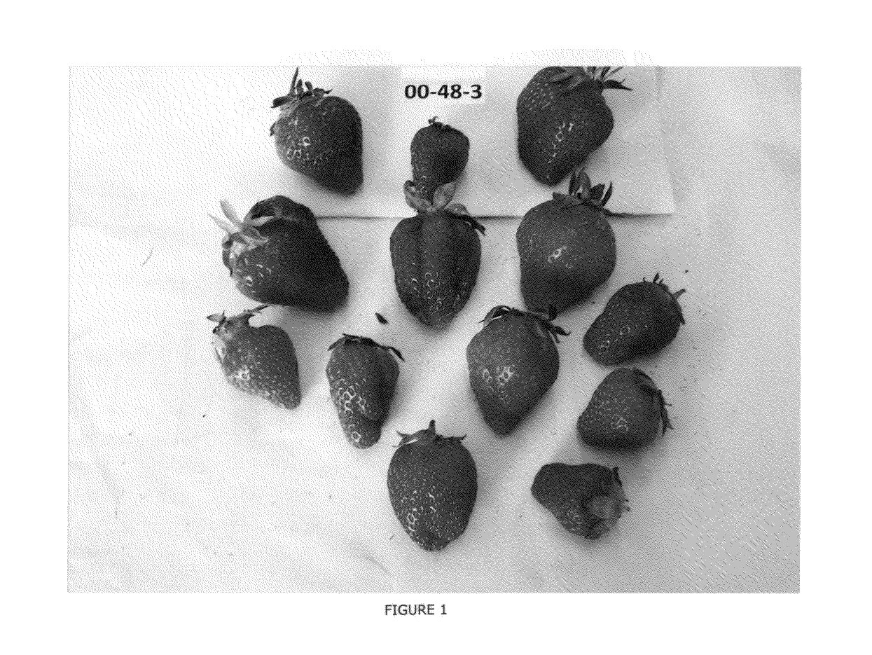

FIG. 2 illustrates the typical size, shape and color of flowers produced by the plant ‘NJ00-48-3’. The flowers are white with 19-22 anthers.

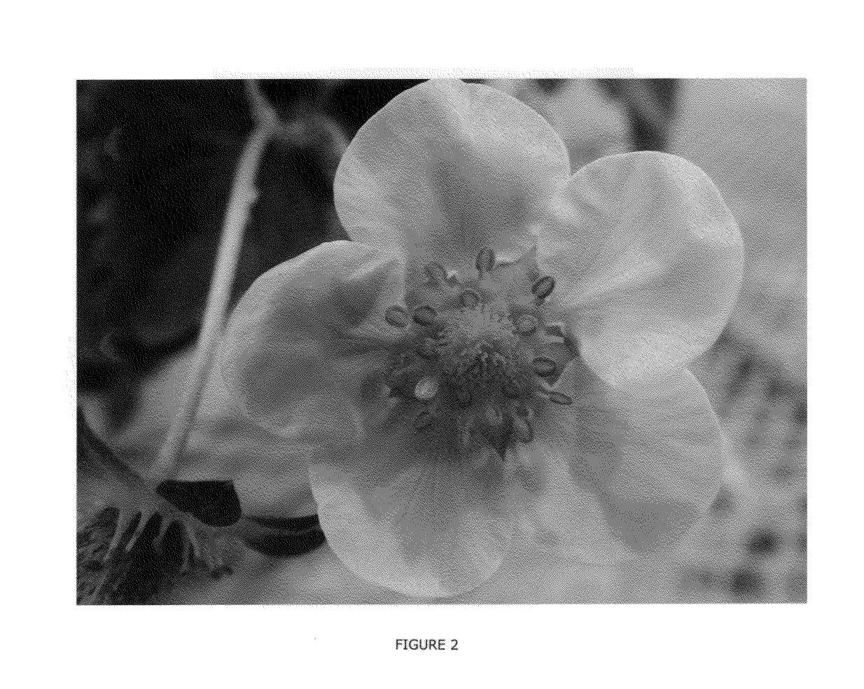

FIG. 3 illustrates the typical branching peduncle formation of ‘NJ00-48-3’ plants, typically forming 4 to 5 branching pedicels that form on each peduncle.

All color references are measured against The Royal Horticultural Society Colour Chart (R.H.S. 1996 Ed.). Colors of foliage, fruit, inflorescence and other plant parts may vary from year to year and from location to location depending on horticultural practices, light conditions, air temperature, soil fertility, etc.

### SUMMARY OF THE VARIETY

The ‘NJ00-48-3’ plant is primarily adapted to the climate and conditions of the eastern United States (zones 5b, 6a & b, 7a & b, 8a & b and 9a) where it demonstrates vigorous, upright plant growth, resulting in an observed tolerance and potential resistance to select common strawberry pathogens. It is characterized by its high production of large, uniformly ripened, conical fruit, with exceptionally sweet and aromatic flavors.

### DETAILED BOTANICAL DESCRIPTION

‘NJ00-48-3’ fruits can be distinguished from other strawberry varieties by a number of distinct characteristics. The primary and secondary fruits are generally conical in shape with an elongated neck, while the tertiary fruits are slightly smaller and are conical in shape without the elongated neck. The sepals are generally uplifted with a distinct fruit neck. The fruits are longer than they are wide with a 2.07:1 length to width ratio. The berries ripen acropetally from the fruit shoulder to the tip. The ripened berries have a very uniformly mild red color RHS 46C with a shiny appearance.

The leaf petioles show upright directional growth rather than prostrate; this leads to the formation of a symmetrical bushy canopy of leaves at the top of the plant. The lobes of the trifoliate leaves have numerous hairs on both the adaxial and abaxial sides.

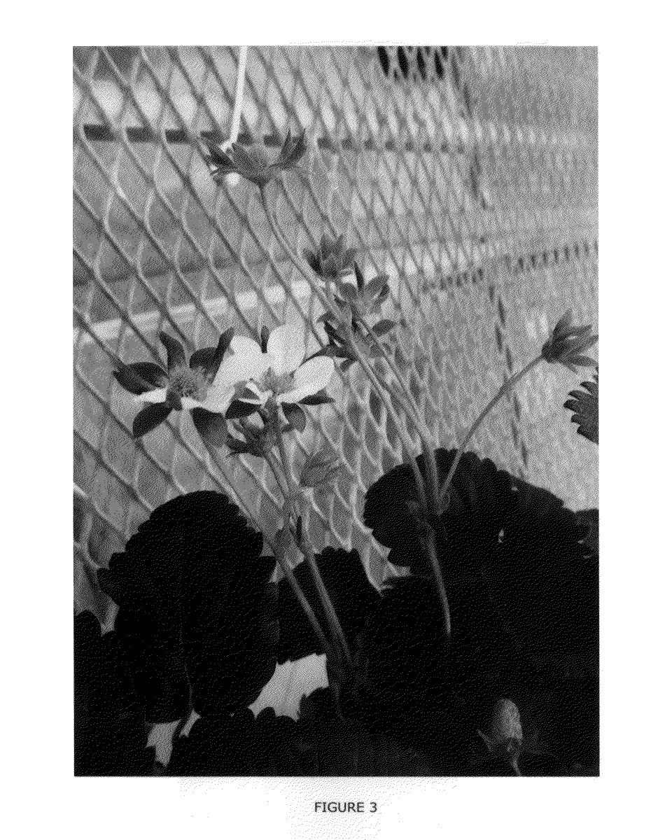

This genotype exhibits the branching type of inflorescences. This structure contains a branching peduncle, (RHC 4D yellow in color) which branches into 4-5 pedicles. Each pedicle bears a single flower and produces a commercial quality berry (see FIG. 3).

The hermaphroditic flowers develop with great regularity, containing 18-22 plump anthers RHC 163A grey orange in color (shown in FIG. 3) loaded with well developed functional pollen grains. The color of the flowers is white RHS 155C

In regard to the pollination process, observations in various seasons and locations of growth have indicated that this process is regular, leading to formation of well-shaped, attractive, commercial quality fruits.

This is a short day variety that produces the majority of its fruit mid- season. From the time of flowering to the time of fruit ripening (harvesting), it normally takes about 28-34 days; which categorizes this genotype as a mid-season variety. Fruits turn red acropetally, from the fruit knack toward the tip. Under particularly warm conditions in the spring, a primary fruit can become completely red in a single day; however, formation of anthogenesis, sugars, aromatics and other quality ingredients require at least two additional days.

TABLE 1
Field performance of NJAES
strawberry selections, Pittstown, NJ 2010

| | | Chandler | Avalon |
|---|---|---|---|
| | ‘NJ00-48-3’ | (PP5,262) | (PPl1,372) |
| Marketable yield (lb/A) | 12,290 b | 11,388 b | 8,523 b |
| Average fruit size (g)^{y} | 20.40 a | 17.09 cd | 16.09 d |
| % Soluble Solids (° Brix) | 9.70 ab | 8.43 de | 8.91 cd |

^{y}Twenty representative fruit/plot
Mean separation within columns by LSD, α = 0.05

TABLE 2
Field performance of NJAES
strawberry selections, North Brunswick, NJ 2011

| | ‘NJ00-48-3’ | Chandler |
|---|---|---|
| Marketable yield (lb/A) | 11,226 a | 10,667 a |
| Average fruit size (g) ^{y} | 12.40 a | 12.60 a |

^{y}Twenty representative fruit/plot
Mean separation within columns by LSD, α = 0.05

TABLE 3
Field Performance of NJAES strawberry
selections and cultivars, Salisbury, NC 2010

| | | | | Florida | |
|---|---|---|---|---|---|
| | | | Camarosa | Radiance | Galletta |
| | ‘NJ00-48-3’ | Chandler | (PP8,708) | (PP20,363) | (PP19,763) |
| Marketable yield (lb/A) | 11,119 b | 14,192 a | 9,692 bc | 7,417 cde | 6,394 de |
| Average fruit size (g)^{y} | 22.7 cd | 21.9 cd | 24.1 bc | 23.0 cd | 26.1 b |
| % Soluble Solids (° Brix)^{x} | 8.31 a | 7.16 cd | 7.68 bc | 5.98 f | 7.75 abc |

^{y}Twenty five representative fruit/plot
Mean separation within columns by LSD, α = 0.05

We claim:

1. A new and distinct variety of strawberry plant designated ‘NJ00-48-3’ substantially as herein shown and described, which is a short day cultivar distinguished by its elongated petioles, upright plant growth and large fruits, with a uniform red color, shiny in appearance and conical in shape with exceptional flavor.
